(19) United States
(12) Patent Application Publication
(10) Pub. No.: US 2024/0067773 A1
(43) Pub. Date: Feb. 29, 2024

(54) ORGANIC CONDUCTING POLYMERS AND USES THEREOF

(71) Applicant: Sai Ming Ngai, Hong Kong (CN)

(72) Inventor: Sai Ming Ngai, Hong Kong (CN)

(21) Appl. No.: 18/374,958

(22) Filed: Sep. 29, 2023

Related U.S. Application Data
(63) Continuation of application No. 16/876,312, filed on May 18, 2020, now abandoned.
(60) Provisional application No. 62/855,301, filed on May 31, 2019.

(51) Int. Cl. C08G 61/12; G01N 27/407

(52) U.S. Cl. CPC C08G 61/124; C08G 61/125; C08G 61/126; G01N 27/4071; G01N 27/4075; C08G 2261/3241; G01N 33/0054

(57) ABSTRACT

This invention relates to compositions of conducting polymers and their producing methods and applications in sensing technology. The present conducting polymer comprises an electron deficient and an electron rich building block in an alternated repeating unit which can function as sensors to detect, qualify or quantify analytes in fluid and exhibit chemiresistive property and stable performance in normal room temperature and air pressure. In one embodiment, the present invention provides compositions of conducting polymers and devices comprising the present compositions or conducting polymers for sensor application. In another embodiment, the present invention provides methods of detecting target molecules using compositions, conducting polymers or devices of the present invention. The target molecules include without limitation volatile organic compounds (VOCs) which are indicative of the presence or stage or a disease, or indicative of a health status of a subject.

### CROSS-REFERENCE TO RELATED APPLICATIONS

This application claims the benefit of U.S. Provisional Application No. 62/855,301, filed May 31, 2019. The entire contents and disclosures of the preceding application are incorporated by reference into this application.

Throughout this application, various publications are cited. The disclosures of these publications in their entireties are hereby incorporated by reference into this application to more fully describe the state of the art to which this invention pertains.

### FIELD OF THE INVENTION

This invention in general relates to organic conducting polymers and their uses in devices and methods for detecting molecules.

### BACKGROUND OF THE INVENTION

Conducting polymer or conjugating polymer (CP) is a polymeric organic substance that has an extended conjugation of π-electrons within the polymer repeating unit. Ordinary polymers, or commonly known as “plastics”, are usually electrical insulators, in which electrons within the organic material are localized in the highest occupied molecular orbital (HOMO) (also known as the valence band (VB)) of the material (FIG. 1). Large amount of energy is required to excite the electrons from the HOMO level to the lowest unoccupied molecular orbital (LUMO) (also known as the conduction band (CB)) of the material to make it electrically conductive. In metals, the conduction band and valence band overlap, and since electrons are free to move in the conduction band, metals are usually conductors. In FIG. 1, the energy levels of insulators, semiconductors and conductors are demonstrated. The energy difference between the two bands is known as the band gap. Different materials may have different band gaps and energy levels. In organic electronics, chemists make use of synthesis techniques to design and create organic electronic materials with various band gaps and energy levels by adding different functional groups to the materials. The functional groups may either be electron-rich or electron-deficient, and this may greatly affect the energy levels for different applications ranging from organic solar cells or organic photovoltaics (OPVs), organic light emitting diodes (OLEDs), organic field-effect transistors (OFETs), batteries, sensors and so on.

Conducting polymers have been applied to various areas such as sensors, optoelectronics, anti-corrosion and electrocatalysis. CPs have a chemiresistive property which means their electrical resistance is sensitive to chemical environment, this allows them to function as sensors and be utilized in various detection technologies such as electronic noses or other detectors. The characteristics of CPs can be further modified by adding different dopants (e.g. iodine, p-Toluenesulfonic acid (pTSA), hydrochloric acid (HCl), hydrobromic acid (HBr), Tetracyanoquinodimethane (TCNQ) and 7,7,8,8-Tetracyano-2,3,5,6-tetrafluoroquinodimethane (F4TCNQ)), and/or by altering their functional groups (e.g. R1, R2 and so on). This expands the scope of potential uses of CPs in multiple areas.

However, there are some major bottlenecks in applying CPs in commercial products broadly. The first is their low solubility in solvents. Some CPs such as polyacetylene, polyaniline, and polypyrrole are not conductive in their organic soluble neutral states and require addition of dopants to achieve workable conductivities, however it will lower their solubility in organic solvents (Zhang, 2006; Bruna, 2018; Mumtaz, 2008). Their production methods such as electropolymerization are not efficient since a large portion of raw materials are usually wasted during the process and also time consuming, the low solubility in organic solvent will further complicate the synthesis process and make it tedious and inefficient. Secondly, CPs have a low stability under room conditions. For instance, poly(3,4-ethylenedioxythiophene) polystyrene sulfonate (PEDOT:PSS) can be synthesized by in-situ doping of the PEDOT polymer by the polymeric electrolyte PSS during the polymerization process. PEDOT, which is a combination of two polymers, may undergo phase segregation in a prolonged period and hence not stable in the long run. The polymer PEDOT:PSS itself also lacks chemical resistance and can be degraded upon repeated sensing processes in room condition where the dopant PSS will be dissolved in water vapors in the air, causing separation from PEDOT. Other types of doped-CP may also exhibit poor ambient stability. For example, if iodine is used as dopant in CP, it will evaporate easily through time under room conditions. For other CPs, the organic backbone is relatively unstable in the presence of atmospheric moisture when the CPs is charged, this significantly lowers limit the life of the fabricated sensor unit. Thirdly, the low selectivity of CPs towards many volatile organic compounds (VOCs) and some non-volatile organic compounds (non-VOCs) such as ammonia gas and hydrogen sulfide gas also limit the potential of commercialization due to the inefficiency and insufficient accuracy to distinguish analytes.

In sensor technology, metal oxides and other semi-metal base materials are commonly used to construct sensors because of the high stability of inorganic materials, the high chemical resistance towards organic analytes, and their simpler synthetic procedures and hence higher accessibility. However, inorganic material-based VOC sensors typically work under high temperature (e.g. 111-650° C.) and may not function well at room temperature. Their manufacturing conditions are harsh, for instance, the fabrication of traditional semi-metal based electronic devices usually require a high temperature and high vacuum during thermal evaporation of metals or metal oxides or other processes, and the use of corrosive and toxic chemicals such as strong acids (e.g. hydrofluoric acid, piranha solutions) and heavy metals. In addition, as organic sensing may be more favorable when organic-organic interactions are present, the lack of organic functional groups generally makes inorganic materials less sensitive and selective towards organic analyte when compared to CPs.

In view of the foregoing, the present invention introduces conducting polymers which have improved properties and are suitable for a wide range of applications.

### SUMMARY OF THE INVENTION

This invention relates to compositions of conducting polymers and their producing methods and applications in sensing technology.

The present invention provides compositions of conducting polymers and devices comprising the present compositions or conducting polymers for sensor application.

The present invention further provides methods of detecting target molecules using compositions, conducting polymers or devices of the present invention. The target molecules include without limitation volatile organic compounds (VOCs) which may be indicative of the presence or stage or a disease, or indicative of a health status of a subject.

### BRIEF DESCRIPTION OF THE FIGURES

FIG. 1 shows the respective energy levels, valence band and conduction band of insulators, semiconductors, and conductors.

FIG. 2 shows the chemical structure of some embodiments of the present conducting polymer.

FIG. 3 is a schematic diagram showing the structure of a sensor with an organic matrix made from the present conducting polymers.

FIG. 4 shows a plot of current (from −0.8 to 0.8 mA) and voltage (from −1.0 to 1.0 V) of Polymer P1 (polymer P1-6 before cleavage and doping).

FIGS. 5A-5C show the curves of normalized current and time (second) of a sensing matrix made from polymer P1 (polymer P1-6 as an example) in response to three molecules (chloroform/CHCl₃, ethanol/EtOH and acetone). The operation voltage was 1V and measurements were taken using a source/measure unit (SMU).

FIG. 6 shows the Principal Component Analysis (PCA) plot of 8 different volatile organic compounds (VOCs) using signal characteristics of the corresponding VOCs measured by sensing material made from the polymer (Polymer P1-6 as an example after side chain cleavage with pTSA as dopant) (˜10% wt dopant in the polymer complex). Principal component 1 represents magnitude of change of signal intensity while principal component 2 represents elapsed time which means the time taken from responding to recovering to normal.

FIG. 7 is a schematic diagram showing the structure of the sensing device for Principal Component Analysis (PCA).

FIG. 8 shows the specific voltage signals produced over time using Polymer P1-6 after side chain cleavage with pTSA as dopant (˜10 wt % dopant in the polymer complex) as the sensor material on acetone, ethanol, formaldehyde and breaths of two different persons. Intensity denotes an arbitrary voltage signal (in reference to a predetermined value ranging from 100 mV to 5V) measured for the tested molecules. The results indicated that P1 can detect unique patterns of voltage varying with time of different molecules.

FIG. 9 shows the change in conductivity of a doped polymer film made from Polymer P1-6 operated under room conditions during a period of 120 days.

FIG. 10 is a schematic diagram showing the circuitry of the sensor.

FIG. 11 is a diagram showing the polymer molecular weights measured using a high temperature size exclusion chromatography (SEC) system at 140° C. using 1,2,4-trichlorobenzene (TCB) as eluent and polystyrene polymers as standards. The weight average molecular weight (Mw) and number average molecular weight (Mn) were found to be 99535 and 17449 Daltons.

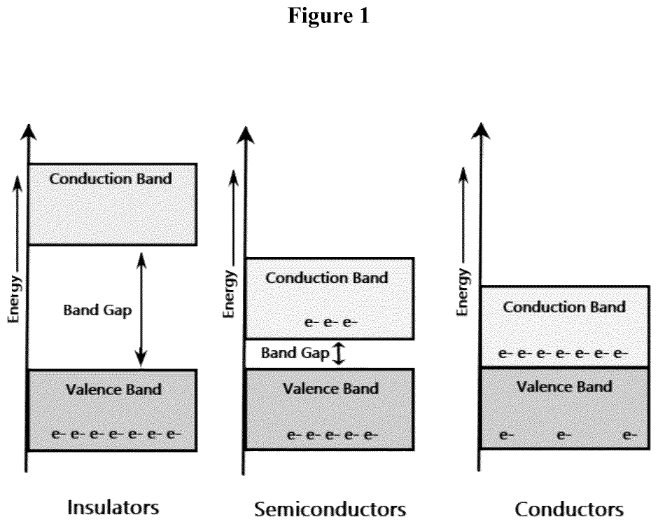

### DETAILED DESCRIPTION OF THE INVENTION

This invention relates to compositions of conducting polymers and their producing methods and applications in sensing technology.

In one embodiment, the present invention provides compositions of conducting polymers and devices comprising the present compositions or conducting polymers for sensor application.

In one embodiment, the present invention provides methods of detecting target molecules using compositions, conducting polymers or devices of the present invention. The target molecules include without limitation volatile organic compounds (VOCs) which may be indicative of the presence or stage or a disease, or indicative of a health status of a subject.

### Conducting Polymers

The present invention provides new classes of conducting polymers which have improved properties, their manufacturing methods and applications. The present conducting polymers have notable advantages over currently available conducting polymers such as improved chemiresistance, thermal and ambient stability, and other electronic and chemical properties, and can be applied to a wide variety of technologies related to sensing or detection of molecules or other electronics aspect such as transistors, solar cells, batteries, anti-static coating, and infrared detector.

The conducting polymer of the present invention is soluble in organic solvents; stable in room temperature, pressure and humidity, and only requires a low working voltage. Therefore, sensors constructed using the present polymer can function at room temperature and requires only very small voltage (1V). Furthermore, its high solubility in organic solvents make the synthesis of the present conducting polymers easier. Fabrication of the sensors using the present conducting polymers is also easier and more flexible. For instance, sensors can be printed on different surfaces using appropriate printing devices, these expand the industrial applications of the present conducting polymers.

In one embodiment, the present conducting polymer comprises thermally or acid cleavable chemical components which is capable of producing a highly cross-linkable thin film of small thickness such as 10-100 nm. The thin film has high chemical resistance and is resistant to various chemicals such as chloroform, acetone, methanol, toluene, chlorobenzene, dichlorobenzene, mineral acids and water.

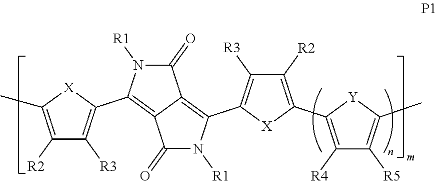

In one embodiment, the present conducting polymer comprises an electron-deficient pigment and an electron-rich aromatic component as a building block. For example, diketopyrrolopyrrole (DPP) pigment body and a simple aromatic body such as phenyl, naphthalenyl, thiophenyl, bithiophenyl, selenophenyl and pyrrolyl group can be used as the building block.

In one embodiment, the current conducting polymer has a general chemical structure represented by Formula P1 (“polymer P1” used herein). The polymer P1 comprises a five-membered ring heterocyclic flanked diketopyrrolopyrrole acceptor building block, having the amide group functionalized by R1. Possible functional groups of R1 include hydrogen, acyl, alkyl, alkenyl, alkynyl, hydroxyalkyl, halogens, haloalkyl, esters, ethers, aldehydes, ketones, carboxylic acids, azos, Z-alkyl and metal containing organometallic complexes such as ferrocenes and porphyrin metal complex, wherein Z can be cyclic ether, amine, amide, imine, azide and sulfonyl. In one embodiment, the Z-alkyl functional group such as amide-alkyl can be further modified in subsequent functionalization of the polymer. The heteroatom X can be an oxygen, sulfur or selenium atom. Possible functional groups of R2, R3, R4 and R5 include hydrogen, alkyl, alkenyl, alkoxy, alkynyl, hydroxyalkyl, halogens, haloalkyl, esters, ethers, cyclic ethers (crown ethers), amines, amides, imines, aldehydes, ketones, carboxylic acids, azides, azos, amide functionalized hydrocarbon chain, and metal containing organometallic complexes such as ferrocenes and porphyrin metal complex. The donor building block comprises a single or multiple linked heterocyclic aromatic building block with any possible integers of n=1-5. The heterocyclic building block may have the heteroatom Y to be an oxygen, sulfur or selenium atom. The donor-acceptor polymer may have repeating units distributed with m=10 or above in order for the material to possess the aforementioned electrical properties. The polymer alone is a semiconductor or a low conductive material.

In one embodiment, a linker or a spacer can be added to the backbone and/or side chains of the present polymer for attaching various functional groups to the polymer. The linker or spacer can be added to the polymer by click chemistry wherein the reactions are high-yielding, wide in scope and create by-product that can be removed without chromatography, including azide-alkyne Huisgen cycloaddition. Any linker or spacer that is compatible with the present polymer can be used.

In one embodiment, a dopant such as elemental iodine, p-toluenesulfonic acid, hydrofluoric acid, hydrochloric acid or hydrobromic acid is added to the present conducting polymer to enhance the conductivity of the polymer. One of ordinary skill in the art would be able to add a dopant to the present conducting polymer according to methods and materials known in the art at the time of this invention (e.g. Macdiarmid, 1987; Schubert, 2005; Bai, 2007). In one embodiment, the dopant used in this invention is p-toluenesulfonic acid or hydrochloric acid, which are easily accessible and inexpensive, have low safety concerns, and require less complicated synthesis in comparison to other compounds.

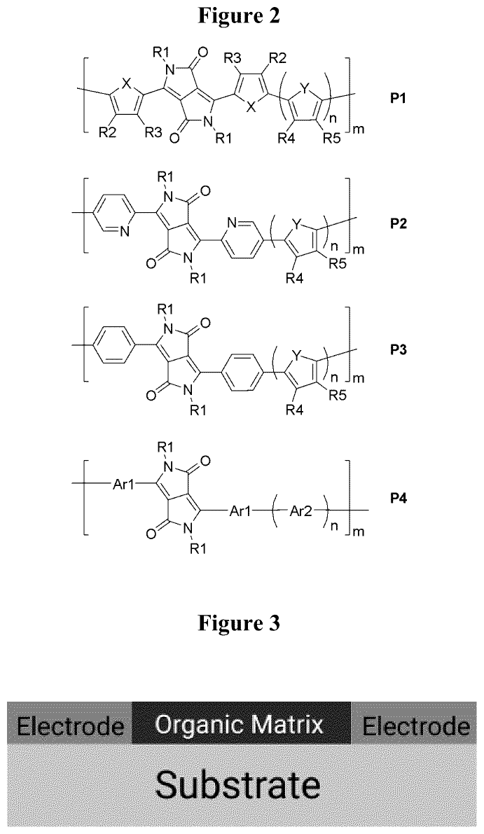

In one embodiment, the present conducting polymer is further functionalized by varying the donor and the acceptor building blocks. The general chemical structures of the final active sensing polymer are represented respectively by Formulae P1, P2, P3 and P4 (FIG. 2). The possible acceptors can be altered using a six-membered aromatic ring on the diketopyrrolopyrrole core such as pyridinyl or phenyl group.

Possible functional groups of R1 include hydrogen, acyl, alkyl, alkenyl, alkynyl, hydroxyalkyl, halogens, haloalkyl, esters, ethers, aldehydes, ketones, carboxylic acids, azos, Z-alkyl and metal containing organometallic complexes such as ferrocenes and porphyrin metal complex, wherein Z can be cyclic ether, amine, amide, imine, azide and sulfonyl. In one embodiment, the Z-alkyl functional group such as amide-alkyl can be further modified in subsequent functionalization of the polymer.

Possible functional groups of R2, R3, R4 and R5 include hydrogen, alkyl, alkenyl, alkoxy, alkynyl, hydroxyalkyl, halogens, haloalkyl, esters, ethers, cyclic ethers (crown ethers), amines, amides, imines, aldehydes, ketones, carboxylic acids, azides, azos, and metal containing organometallic complexes such as ferrocenes and porphyrin metal complex.

In one embodiment, R1 can be a long chain hydrocarbon, an acyl group, a carboxyl group or a hydrogen atom, wherein the long chain hydrocarbon can have 10-25 carbons.

In one embodiment, R2 and R3 can be a hydrogen atom, a straight saturated hydrocarbon chain, or a branched saturated hydrocarbon chain.

In one embodiment, R4 and R5 can be a hydrogen atom, a straight saturated hydrocarbon chain, a branched saturated hydrocarbon chain, an alkoxy chain, an ester, an ether, or an amide functionalized hydrocarbon chain.

In one embodiment, the aromatic components of the donor or acceptor building blocks can be further extended to fused aromatic (AR) systems. For example, AR1 and/or AR2 in Polymer P4 can be naphthalenyl, anthracenyl, phenanthracenyl, triphenylene, pyrenyl, thienothiophenyl, dithienothiophenyl and benzodithiophenyl.

### Methods for Producing the Present Conducting Polymers

In one embodiment, the synthesis of the present conducting polymer comprises the following steps:
(a) Preparing a diketopyrrolopyrrole (DPP) acceptor building block with electron deficient amide side chains;
(b) Functionalizing a pyrrole nitrogen of the DPP acceptor building block;
(c) Treating the product of step (b) with a brominating reagent, thereby obtaining a brominated product;
(d) Preparing a donor building block capable of undergoing Stille-type reaction with a brominated compound; and
(e) Allowing the brominated product of step (c) and the donor building block of step (d) to undergo Stille-type reaction, thereby obtaining the conducting polymer.

In one embodiment, the synthesis of DPP acceptor building block for P1 comprises following steps (using compounds 1 and 2 to illustrate):

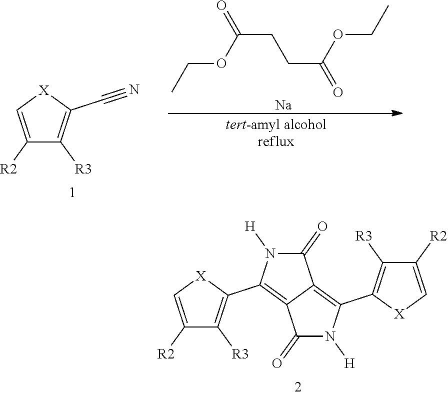

(a) Dissolving sodium in tert-amyl alcohol in a two-necked round bottom flask connected to nitrogen atmosphere, wherein one neck of the flask is connected to a condenser and the other neck is stopped by a rubber septum;
(b) Adding diethyl succinimide slowly into the solution of step (a) by a syringe via the rubber septum;
(c) Dissolving a cyano containing heteroaromatic compound 1 (X can be 0, S or Se) in small amount of tert-amyl alcohol and injecting into the reaction mixture by a syringe via the rubber septum in 30 minutes;
(d) Refluxing the reaction for 24 hours;
(e) Adding acetic acid and methanols sequentially to precipitate the product; and
(f) Filtering and washing the precipitation with water and methanol to obtain compound 2.

In one embodiment, functionalization of pyrrole nitrogen of the DPP acceptor building block comprises the following steps (using compounds 2 and 3 to illustrate):

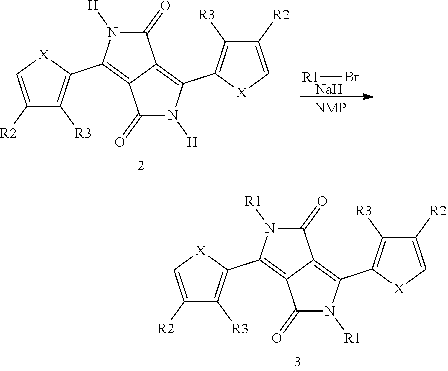

(a) Dissolving compound 2 in anhydrous NMP in a round bottom flask;
(b) Adding sodium hydride into the solution and stirring the reaction mixture at room temperature under nitrogen for 1 hour;
(c) Adding R1-Br into the solution slowly and stirring at room temperature for 18 hours;
(d) Quenching the reaction mixture slowly with deionized water of room temperature;
(e) Using liquid-liquid extraction to extract the crude product with chloroform, ethyl acetate, diethyl ether or toluene and washing it successively with water and brine, wherein the organic portion is collected, and solvent is removed; and
(f) Further purifying the crude product by flash column chromatography at room temperature using unmodified silica gel as the stationary phase and chloroform, hexane ethyl acetate, diethyl either or toluene as eluent to obtain compound 3.

In one embodiment, bromination of functionalized compound of the DPP acceptor building block comprises the following steps (using compounds 3 and 4 to illustrate):

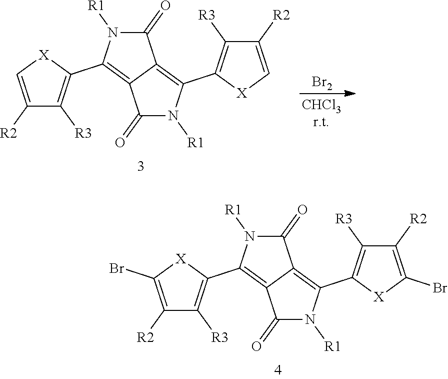

(a) Dissolving compound 3 in chloroform, wherein the solution is purged with nitrogen for 10 minutes;
(b) Adding bromine slowly using a dropping funnel into the reaction mixture at room temperature under nitrogen, and stirring the reaction mixture for an addition of 1 hour;
(c) Quenching the reaction mixture by adding sodium sulfite solution of room temperature;
(d) Using liquid-liquid extraction to extract the product with chloroform and washing it with water to remove the solvent; and
(e) Recrystallizing the product in chloroform/methanol solution to obtain compound 4.

In one embodiment, preparation of a donor building block capable of undergoing Stille-type reaction with a brominated compound comprises the following steps (using compounds 5 and 6 to illustrate):

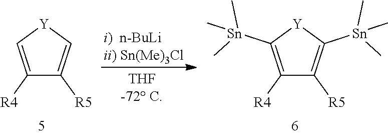

(a) Dissolving compound 5 in anhydrous tetrahydrofuran in a two-necked round bottom flask, wherein one neck of the flask is connected to nitrogen and the other neck is stoppered with a rubber septum.
(b) Cooling down the solution to −72° C. and adding n-butyllithium slowly into the solution using a syringe through the rubber septum.
(c) Stirring the mixture at around −72° C. for 1 hour.
(d) Adding trimethyltin (IV) chloride slowly into the solution through the rubber septum and stirring the reaction mixture at room temperature for 18 hours, wherein water is added to the mixture;
(e) Extracting the crude product with diethyl ether and washing it with hydrochloric acid, wherein the organic portion is then collected and solvent is removed; and
(f) Recrystallizing the crude product in chloroform/methanol to obtain compound 6.

In one embodiment, the Stille-Type reaction for obtaining Polymer P1 comprises the following steps (using compounds 4 and 6 to illustrate):

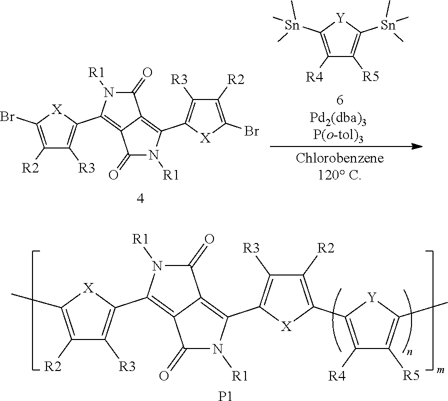

(a) Adding compound 4, compound 6 and Tris(o-tolyl)phosphine at same mole into a two-necked round bottom flask connected to argon atmosphere, wherein one neck of the flask is fitted with a condenser and the other neck is stoppered with a rubber septum, and the flask then undergoes a vacuum-purge cycle for three times and is kept under argon atmosphere;
(b) Adding anhydrous chlorobenzene and Tris(dibenzylideneacetone)dipalladium(0) sequentially;
(c) Stirring the mixture at 120° C. for 24 hours;
(d) Upon cooling to room temperature, pouring the mixture into methanol under stirring; and
(e) Collecting the precipitate and purifying it by Soxhlet extraction using methanol, acetone and chloroform to obtain polymer P1.

Polymers P2, P3 and P4 can be synthesized using similar procedures except the monomers used are different building blocks of donors and acceptors. Examples of donors include thiophene, alkylthiophenes, alkoxythiophenes, bithiophene, thienothiophene, benzodithiophene, carbazole, furan, pyridine, pyrrole, selenophene and benzene, etc.

Extraction methods and solvents described herein for preparing various intermediate compounds and final compounds are not meant to be limiting. A person having ordinary skill in the art would be able to choose appropriate methods and materials known in the art to extract particular compounds with desired yield and purity.

### Doping Agent

In one embodiment, the present conducting polymer has an electronic conductivity ranging from 10′ to 10'S/cm. In one embodiment, the present conducting polymer itself is an ohmic chemiresistive material at room temperature.

The conductivity of the present conducting polymer can be modulated by adding a dopant (or doping agent).

In one embodiment, dopants can be any acidic species such as hydrofluoric acid, hydrochloric acid, hydrobromic acid, hydroiodic acid, p-toluenesulfonic acid, acetic acid, nitric acid, sulfuric acid, phosphoric acid, tetracyanoquinodimethane(TCNQ) and 7,7,8,8-tetracyano-2,3,5,6-tetrafluoroquinodimethane (F4TCNQ).

In one embodiment, one or more dopants are added to the present conducting polymer to alter its electrical properties. The band structure of the doped conducting polymer may vary with the length of side chains of the polymer (e.g. hydrocarbon chains with different lengths). The length may alter the π-stacking distance and the interlamellar distance of each polymer chain. The repeating unit cell would then change and would be reflected as a shift in X-ray Powder Diffraction (XRD) pattern. The alteration of side chains can incorporate different functionality.

By acid doping, the CP of the present invention become more conductive. Presence of bulky functional groups on the side chain may increase the distance between chain to chain, and facilitate the acid to diffuse into the backbone. In one embodiment, the acid doping process of non-side-chain packed polymer takes around 30 minutes for acid to diffuse into the polymer backbone. In one embodiment, the acid doping process of the polymer, wherein the side chains of the polymer are modified with bulky functional groups, takes less than 30 minutes for acid to diffuse into the polymer backbone.

In one embodiment, dopant is an acid such as hydrofluoric acid, hydrochloric acid, hydrobromic acid, hydroiodic acid, p-toluenesulfonic acid, acetic acid, nitric acid, sulfuric acid, phosphoric acid. In one embodiment, the molarity of the acid used is 1-8 M. In one embodiment, the content of the dopant within the complex ranges from 10-50 wt %.

In one embodiment, non-acid dopants are used. In one embodiment, elemental iodine is used as a dopant.

In one embodiment, the acid-doped polymer, wherein the side chains of the polymer are modified with bulky functional groups, can be applied in the fabrication of a pH or ionic sensor.

The present conducting polymer can be stabilized by using appropriate type of dopant, concentration of the dopant, functional groups of the conducting polymer or thermal annealing process so that the conducting polymer exhibits a high lying HOMO energy level and a narrow band gap to form a stable polymer-dopant complex for long-term stability.

### Characterization of Conducting Polymer

The present conducting polymer can be analyzed by their physical and chemical characteristics using techniques known in the art including Nuclear magnetic resonance (NMR) spectroscopy and X-ray spectroscopy.

### Advantages of Conducting Polymer

Since the intermediate compounds for making the present conducing polymer as well as the final conducing polymer are soluble in organic solvents, manufacturing of the present conducing polymer is much simpler and more cost-effective than the manufacturing of conventional conducting polymers.

The present conducting polymer exhibits ambient stability since it does not prone to degradation under room conditions (i.e., ambient temperature, humidity and pressure). It also has a high chemical resistance and hence remains stable against various chemical agents. This gives a high durability and robustness of the sensors made from the present conducting polymer.

Furthermore, the present conducting polymer is able to work at low voltage and hence sensors comprising the present conducting polymer only require a low level of operating power.

Overall, the present conducting polymer has advantages over metal oxides since it can operate under room temperature as oppose to high temperature (e.g. 100-600° C.). It has a wider versatility since its selectivity towards molecules can be engineered by modifying the side chains of the polymer and/or coupling with dopant agent. Since the polymer can be engineered to be soluble in various solvents, such a feature allows one to fabricate molecularly imprinted sensor through solution processing. The side chains can also be substituted by analyte-recognizing moieties. When the sensor is in operation, the analyte may alter the bulk morphology of the conductive matrix, causing a selective response.

On the other hand, the present conducting polymer has advantages over conventional conducting polymers for its higher stability at room conditions. Its shelf life is longer (>4 months) and durability is higher. Its higher solubility in organic solvent make its fabrication easier since it can be printed on various surface. Together with its low operating power requirement and side chain flexibility, the present conducting polymer is suitable for commercialization and various sensor applications.

### Conductive Ink

The present conducting polymer is soluble in a wide range of organic solvents and can be used to prepare conductive ink comprising the organic solvent and the conducting polymer.

In one embodiment, the present conducting polymer can be dissolved in common organic solvent such as chloroform, chlorobenzene, dichlorobenzene, toluene, nitrobenzene, ethyl acetate, xylenes to give a conductive ink solution. The conductive ink may comprise other additives to stabilize the ink for longer shelf life if needed. In one embodiment, the concentration of the CP in organic solvents ranges between 1-10 mg/mL.

The conductive ink can then be applied to any surface such as a sensor substrate easily. Conducting polymer in the conductive ink will deposit on the substrate and the solvent will evaporate leaving the conducting polymer on the substrate surface. Thickness of the conducting polymer on the sensor surface can be adjusted manually. Thermal annealing can be carried out to make the deposited conducting polymer thin film highly insoluble and hence more durable.

In one embodiment, the R1 side-chain on the polymer first acts as a solubilizing side chains to form a conductive ink in a solvent. In one embodiment, once the polymer, where the R1 side-chain is acyl, carboxyl, methyl, ethyl, or other thermally labile functional group, is deposited onto the substrate by solution processing techniques (e.g. spin-coating, blade coating or inkjet printing), the side chain can be thermally removed at elevated temperature to release an N—H bond on the acceptor chromophore which enables an insoluble thin film layer to be formed with enhanced stability and good chemical resistance. In other embodiments, the side-chain is acid-cleavable. In one embodiment, the thermal annealing process takes at least 20 minutes to ensure full cleavage of side chains. In one embodiment, the thermal annealing process takes at least 30 minutes.

In one embodiment, the substrate is glass, silicon dioxide, ceramics, polyethylene terephthalate (PET) or other plastic substrate.

### Applications of the Present Invention

The present conducting polymers are capable of detecting particular types of molecules and can be used to produce sensing materials and sensors for detection of specific types of molecules. The sensing materials and sensors can be of various configurations and kinds depending on the nature and abundance of molecules to be detected. The present invention can be adapted for research, industrial or clinical uses, covering various disciplines such as biomedical and environmental.

In one embodiment, the present conducting polymers and sensors detect molecules in liquids or gases. The liquid and gases can be obtained from living organisms, environment, synthetic samples or laboratory samples.

In one embodiment, the target molecules to be detected are present in biological samples such as breathe, blood, plasma, serum, urine, saliva, fecal matters, bodily discharges and cell culture of any kind.

In one embodiment, the present conducting polymers and sensors are used for biomedical applications such as the detection of a molecule that is indicative of the presence or stage of a disease, or indicative of a health status of a subject. The molecule can be a biomarker that is associated with the disease or health status in question.

In one embodiment, the present conducting polymers and sensors detect and quantify one or more target molecules in a sample. In one embodiment, the present conducting polymers and sensors distinguish a target molecule from other non-targets in a mixture of molecules.

In one embodiment, the present invention is used in environmental toxicology for the detection of toxins or metal ions in water or toxic gases in atmospheric air. This helps monitor the level of these molecules in water and air and evaluate the effects on living organisms in the affected areas.

### Gaseous Molecules

In one embodiment, the sensors made from the present conducting polymer are able to detect the presence of two or more gaseous molecules at the same time. In one embodiment, the sensors made from the present conducting polymer are able to differentiate different gaseous molecules.

In one embodiment, the sensors made from the present conducting polymer are able to generate signals in proportional to the quantity of the target gaseous molecules and thereby quantify the gaseous molecules.

The sensors made from the present conducting polymer can detect a target type of gaseous with high sensitivity. In one embodiment, the limit of detection is about 100 ppb or above. In one embodiment, the sensors made from the present conducting polymer can detect gaseous molecules with a limit of detection of about 1000 ppb under a well-controlled gas flow system (i.e., a constant carrier gas flow rate, humidity and temperature).

In one embodiment, the gaseous molecules are volatile organic compounds (VOCs).

VOCs have been reported for its association with certain diseases and used as biomarkers for screening of diseases. For example, acetone is a VOC being used for diagnosis of diabetes (Rydosz, 2018; Righettoni, 2010; Minh Tdo, 2012). Gastric cancer-specific VOCs were reported and may be used for early diagnosis and prognosis of gastric cancer (Zhang, 2014). Sensors which are selective to VOCs that are biomarker in nature can be used in research and clinical sectors for investigating the diseases in question.

In one embodiment, molecules to be detected by the present invention include but are not limited to the following VOCs: methanol, ethanol, propan-1-ol, isopropanol, acetone, acetic acid, chloroform, hexane, ethyl acetate, toluene, chlorobenzene, diethyl ether, tetrahydrofuran, 1,2-dioxane, 1,4-dioxane, ethyl formate, butanone, acetonitrile, benzene, and carbon disulfide.

In one embodiment, the target molecules to be detected by the present invention are non-volatile organic compounds (non-VOCs). In one embodiment, the target molecules to be detected by the present invention are toxic gases or molecules, including but not limited to formaldehyde, carbon monoxide, ammonia, hydrochloric acid, chlorine, sulfur oxides, and sulfuric acid.

### Metal Ions in Water

In one embodiment, the sensors made from the present conducting polymer are able to detect and/or quantify molecules in liquids such as metal ions in water.

In one embodiment, the backbone or the side chains of the present conducting polymer comprise metal sensitive chemical function groups including but not limited to ethers, amines, amide, imines, Schiff bases, pyridine derivatives, phosphines or other possible ligands.

### Fabrication of Sensors

In one embodiment, the present conducting polymer is fabricated as the active conducting layer in a chemiresistive sensor device, the device can be used to detect molecules in liquids (e.g. metal ions in water) or gases (e.g. volatile organic compounds (VOCs) in a controlled gas flow system) qualitatively or quantitatively, or both. In one embodiment, the sensor comprises one or more types of conducting polymer. In one embodiment, for analyzing a liquid analyte, the sensor is incubated in a solution of the analyte. The analysis can comprise a simple current versus time plot, or by an electrochemical voltammetric scanning with the use of a reference electrode.

In one embodiment, the sensor comprises one or more layer of thin film made from the present conducting polymer. In one embodiment, the thin film used for liquid detection is 30-100 nm thick. In one embodiment, the thin film used for gas detection is 10-100 nm thick.

In one embodiment where the present conducting polymer is used to fabricate a sensor for detection of VOCs, other components can be provided to form a sensing device or system to handle the entire process from sampling, pre-treatment of samples, detection of signals, analysis of signals, control of moisture level and flow rate of gas and the like. For example, filters can be used to remove dusts and other impurities from the sample; sample concentrator can be used to extract the gaseous molecules from a diluted sample and output a concentrated sample; a moisture absorption filter can be used to remove excessive water vapor from the system; and a gas flow rate meter can be used to monitor and control the flow rate of gas within the system (Rydosz, 2018).

FIG. 10 shows one embodiment of the circuitry of the present sensor comprising an operational amplifier and microcontroller. In one embodiment, an operational amplifier is connected to the sensor and amplifies the sensing signal before feeding into a microcontroller.

The invention will be better understood by reference to the Experimental Details which follow, but those skilled in the art will readily appreciate that the specific experiments described are only for illustrative purpose and are not meant to limit the invention as described herein, which is defined by the claims that follow thereafter.

In one embodiment, the present invention provides a polymer represented by a formula selected from the group including

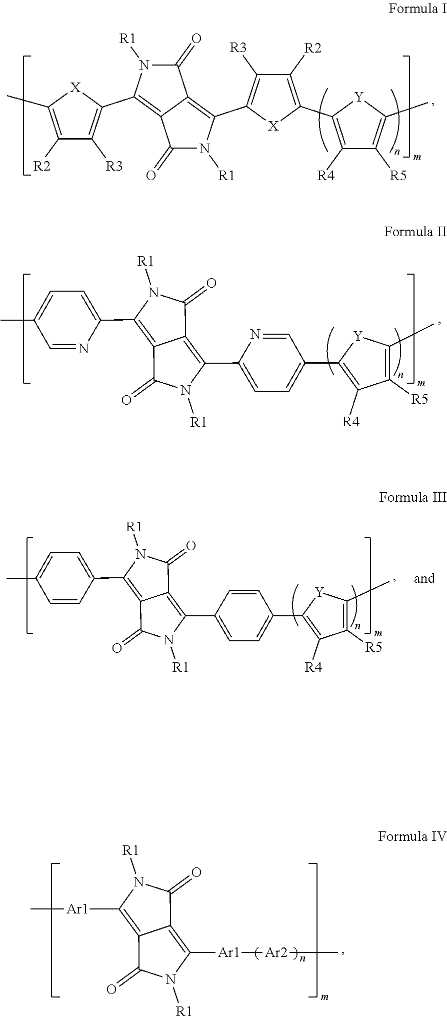

wherein R1 is selected from the group including hydrogen, acyl, carboxyl, alkyl, alkenyl, alkynyl, hydroxyalkyl, halogen, haloalkyl, ester, ether, aldehyde, ketone, carboxylic acid, azo, Z-alkyl, and metal containing organometallic complex, wherein Z is selected from the group including cyclic ether, amine, amide, imine, azide and sulfonyl, wherein each of the R2, R3, R4 and R5 is independently selected from the group including hydrogen, acyl, carboxyl, alkyl, alkenyl, alkynyl, alkoxy, hydroxyalkyl, halogen, haloalkyl, ester, ether, cyclic ether, amine, amide, imine, aldehyde, ketone, carboxylic acid, azide, azo, amide functionalized hydrocarbon chain, and metal containing organometallic complex,
wherein X and Y are independently selected from oxygen, sulphur and selenium;
wherein each of Ar1 and Ar2 is independently selected from the group including naphthalenyl, anthracenyl, phenanthracenyl, triphenylene, pyrenyl, thienothiophenyl, dithienothiophenyl and benzodithiophenyl, and
wherein n ranges from 1-5, and m is at least 3.

In one embodiment, R1 is selected from the group including hydrogen, acyl, long chain alkyl, long chain alkenyl and long chain alkynyl, R2 and R3 are independently selected from the group including hydrogen, straight alkyl chain and branched alkyl chain, and R4 and R5 are independently selected from the group including hydrogen, straight alkyl chain, branched alkyl chain, ester, ether, and amide functionalized alkyl, alkenyl, and alkynyl chain

In one embodiment, n ranges from 1-5, and m is in a range of 10-300. In one embodiment, the Mn of the polymer is in a range of 2,000-100,000 Daltons. In one embodiment, the Mw of the polymer is in a range of 5,000-300,000 Daltons.

In one embodiment, the present invention provides a method of synthesizing the polymer, comprising the steps of
(a) Preparing an acceptor building block comprising a diketopyrrolopyrrole (DPP);
(b) Functionalizing a pyrrole nitrogen of the acceptor building block;
(c) Treating the product of step (b) with a brominating reagent, thereby obtaining a brominated product;
(d) Preparing a donor building block capable of undergoing Stille-type reaction with a brominated compound; and
(e) Allowing the brominated product and the donor building block to undergo Stille-type reaction, thereby obtaining the polymer.

In one embodiment, the present invention provides a method of preparing a thin film on a substrate for a sensing component, comprising the steps of:
a) Printing a solution of the polymer in an organic solvent onto a substrate;
b) Drying the printed solution, leading to a dried film on the substrate;
c) Thermally annealing the dried film; and
d) Submerging the dried film on the substrate in a solution of dopant, thereby obtaining the thin film deposited onto the substrate.

In one embodiment, the organic solvent includes but is not limited to chloroform, chlorobenzene, dichlorobenzene, toluene, anisole or ketone and the solution of the polymer has a concentration of 1-10 mg/mL.

In one embodiment, the printed solution is dried at 100° C.

In one embodiment, the dried film is thermally annealed at 80° C.-250° C. for 10-60 minutes to remove the cleavable side chains R1 from the polymer and form a crosslinked structure which makes the thin film insoluble, stable, durable and chemical resistant.

In one embodiment, R1 is acyl, carboxyl or any other thermally labile functional groups including short chain alkyl groups such as methyl or ethyl.

In one embodiment, the dopant is selected from the group including hydrofluoric acid, hydrochloric acid, hydrobromic acid, hydroiodic acid, p-toluenesulfonic acid (pTSA), acetic acid, nitric acid, sulfuric acid, phosphoric acid, and elemental iodine.

In one embodiment, the dried film on the substrate is submerged in the solution of dopant at room temperature followed by an elevated temperature at 50° C.-80° C.

In one embodiment, the thin film has a thickness of 10-100 nm.

In one embodiment, the present invention provides a device for detecting molecules, comprising a sensing component comprising two or more electrodes on a substrate deposited with a thin film prepared by the method disclosed herein.

In one embodiment, the device further comprises one or more of a) dust filters; b) moisture absorption filters; c) sample concentrator; d) gas flow rate meter; and e) liquid flow meter.

In one embodiment, the device can be operated under room temperature, pressure and humidity and requires a low working voltage.

In one embodiment, the working voltage of the device is 10 mV-10V.

In one embodiment, the polymer of the sensing component has a shelf life of at least 4 months under room temperature, pressure, and humidity.

In one embodiment, the device can detect an analyte with a limit of detection of about 1000 ppb.

In one embodiment, the present invention provides a method of detecting target molecules in a gas or liquid sample using the device, comprising the following steps:
(a) Introducing the sample to the device and conducting a current or voltage scanning, obtaining one or more graphs;
(b) Extracting signal features from said graphs, wherein said features are selected from the group including signal intensity, elapsed time, peak height, peak area, peak FWHM, recovery time, and peak symmetry;
(c) Projecting said features to a map with two or more dimensions, wherein each dimension corresponds to one feature, and the position of a specific compound in said map is unique; and
(d) Determining whether the target molecules are present in the sample.

In one embodiment, the map is a two-dimensional map, one dimension represents elapsed time, and the other dimension represents signal intensity.

In one embodiment, the target molecules are volatile organic compounds (VOCs) and said VOCs are selected from the group including methanol, ethanol, propan-1-ol, isopropanol, acetone, acetic acid, chloroform, hexane, ethyl acetate, toluene, chlorobenzene, diethyl ether, tetrahydrofuran, 1,2-dioxane, 1,4-dioxane, ethyl formate, butanone, acetonitrile, benzene, and carbon disulphide.

In one embodiment, the molecules are selected from the group including formaldehyde, carbon monoxide, ammonia, hydrochloric acid, chlorine, sulphur oxides, and sulfuric acid.

In one embodiment, the method further determines the quantity or concentration of the target molecules in the sample.

Throughout this application, various references or publications are cited. Disclosures of these references or publications in their entireties are hereby incorporated by reference into this application in order to more fully describe the state of the art to which this invention pertains. It is to be noted that the transitional term “comprising”, which is synonymous with “including”, “containing” or “characterized by”, is inclusive or open-ended and does not exclude additional, un-recited elements or method steps.

### EXAMPLES

### Example 1—Preparation of Diketopyrrolopyrrole (DPP) Acceptor Building Block

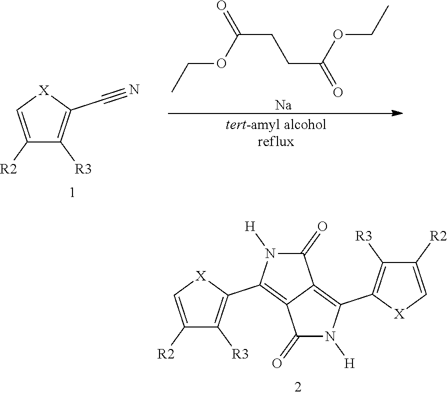

The synthesis of DPP acceptor building block for P1 comprised the following steps:
(a) Dissolving sodium (4.0 mole) in tert-amyl alcohol in a two-necked round bottom flask connected to nitrogen atmosphere, wherein one neck of the flask was connected to a condenser and the other neck was stopped by a rubber septum;
(b) Adding diethyl succinimide (0.5 mole) slowly into the solution of step (a) by a syringe via the rubber septum;
(c) Dissolving a cyano containing heteroaromatic compound 1 (X can be 0, S or Se) (1.0 mole) in small amount of tert-amyl alcohol and injecting into the reaction mixture by a syringe via the rubber septum in 30 minutes;
(d) Refluxing the reaction for 24 hours;
(e) Adding acetic acid and methanol sequentially to precipitate the product; and
(f) Filtering and washing the precipitation with water and methanol to obtain compound 2 with a yield of 40%.

### Example 2 Preparation of Compound 3 with Functionalization of Pyrrolo Nitrogen

Functionalization of pyrrolo nitrogen of compound 3 comprised the following steps:
(a) Dissolving compound 2 (1.0 mole) in anhydrous NMP in a round bottom flask;
(b) Adding sodium hydride (2.2 mole) into the solution and stirring the reaction mixture at room temperature under nitrogen for 1 hour;
(c) Adding R1-Br (3.0 mole) into the solution slowly and stirring at room temperature for 18 hours;
(d) Quenching the reaction mixture slowly with deionized water of room temperature;
(e) Extracting the crude product with chloroform and washing it successively with water and brine, wherein the organic portion was collected, and solvent was removed; and
(f) Further purifying the crude product by flash column chromatography at room temperature using unmodified silica gel (200-450 mesh) as the stationary phase and hexane:ethyl acetate=3:1 as eluent to obtain compound 3 with a yield of 43%.

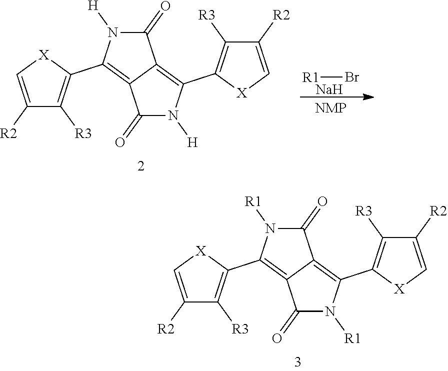

### Example 3—Preparation of Compound 4 (Bromination)

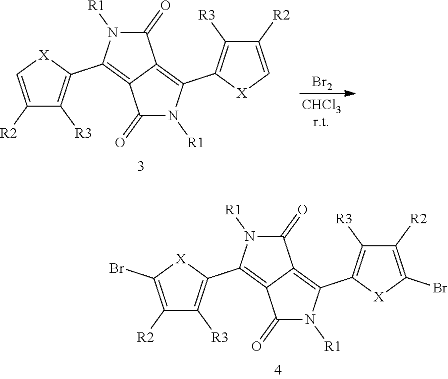

The bromination of compound 3 comprised the following steps:
(a) Dissolving compound 3 (1.0 mole) in chloroform, wherein the solution was purged with nitrogen for 10 minutes;
(b) Adding bromine (2.1 mole) slowly using a dropping funnel into the reaction mixture at room temperature under nitrogen, and stirring the reaction mixture for an addition of 1 hour;
(c) Quenching the reaction mixture by adding sodium sulfite solution of room temperature;
(d) Extracting the product with chloroform and washing it with water, wherein the solvent was removed; and
(e) Recrystallizing the product in chloroform/methanol solution to obtain compound 4 with a yield of 88%.

### Example 4—Preparation of Donor Building Block (Lithiation Followed by S_{N}2)

The lithiation of donor building block comprised the following steps:
(a) Dissolving compound 5 (1.0 mole) in anhydrous tetrahydrofuran in a two-necked round bottom flask, wherein one neck of the flask was connected to nitrogen and the other neck was stoppered with a rubber septum.
(b) Cooling down the solution to −72° C. and adding n-butyllithium (2.0 moles) slowly into the solution using a syringe through the rubber septum.
(c) Stirring the mixture at around −72° C. for 1 hour.
(d) Adding trimethyltin (IV) chloride slowly into the solution through the rubber septum and stirring the reaction mixture at room temperature for 18 hours, wherein water was added to the mixture;
(e) Extracting the crude product with diethyl ether and washing it with 0.1N hydrochloric acid, wherein the organic portion was then collected and solvent was removed; and
(f) Recrystallizing the crude product in chloroform/methanol to obtain compound 6 with a yield of 62%.

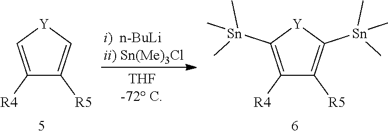

### Example 5—Preparation of Polymer P1 (Stille-Type Synthesis)

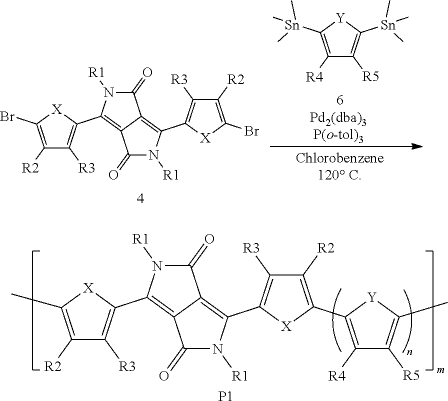

In one embodiment, R1 in Polymer P1 is tert-butyloxycarbonyl, R2 and R3 are hydrogen, R4 and R5 are methoxy, and X and Y are sulfur. See Polymer P1-1 and Polymer P1-2 in Table 1. For illustrative purpose only and not meant to be limiting the invention, additional Examples of P1 are listed in Table 1 wherein X and Y are sulfur.

The Stille-Type Synthesis of Polymer P1 comprised the following steps:
(a) Adding compound 4 (1.0 mole), compound 6 (1.0 mole) and Tris(o-tolyl)phosphine (0.001 mole) into a two-necked round bottom flask connected to argon atmosphere, wherein one neck of the flask was fitted with a condenser and the other neck was stoppered with a rubber septum, and the flask was then undergone a vacuum-purge cycle for three times and kept under argon atmosphere;
(b) Adding Anhydrous chlorobenzene and Tris(dibenzylideneacetone)dipalladium(0) (0.001 mole) sequentially;
(c) Stirring the mixture at 120° C. for 24 hours;
(d) Upon cooling to room temperature, pouring the mixture into methanol under stirring; and
(e) Collecting the precipitate and purifying it by Soxhlet extraction using methanol, acetone and chloroform to obtain polymer P1 with a yield of 92%.

TABLE 1
Typical Polymers with Similar Structure

| Polymer | R1 | R2 | R3 | R4 | R5 | n | m |
|---|---|---|---|---|---|---|---|
| P1-1 | t-Boc | H | H | OMe | OMe | 1 | ≥10 |
| P1-2 | t-Boc | H | H | OMe | OMe | 2 | ≥10 |
| P1-3 | t-Boc | H | H | H | OMe | 2 | ≥10 |
| P1-4 | t-Boc | H | H | OMe | H | 2 | ≥10 |
| P1-5 | t-Boc | H | H | OEt | OEt | 1 | ≥10 |
| P1-6 | t-Boc | H | H | OEt | OEt | 2 | ≥10 |
| P1-7 | t-Boc | H | H | H | OEt | 2 | ≥10 |
| P1-8 | t-Boc | H | H | OEt | H | 2 | ≥10 |

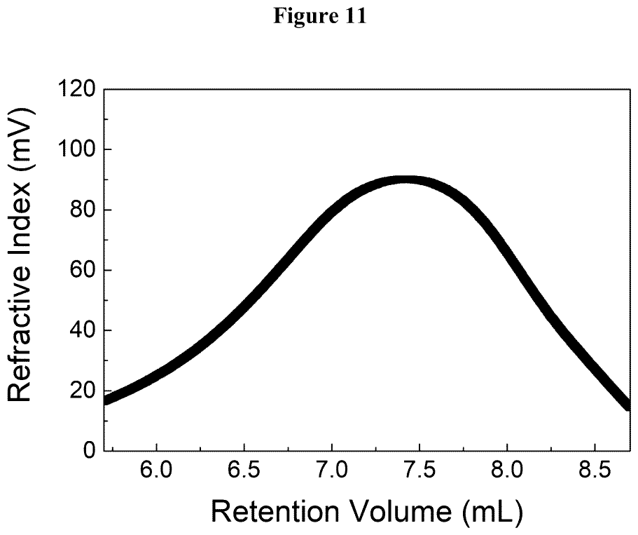

In one embodiment, the synthesized polymers have a number average molecular weight at least 2000 Daltons. In one embodiment, the synthesized polymers with m≥10 (see Table 1) were used for the sensor with better performance than those with m<10. In one embodiment, as shown in FIG. 11, a representative polymer P1-6 has a weight average molecular weight (Mw) of 99535 Daltons and number average molecular weight (Mn) of 17449 Daltons as measured by GPC with a refractive index detector at 140° C. using 1,2,4-trichlorobenzene as eluent and polystyrene polymers as standards. The polydispersity index (PDI) is 5.70. In one embodiment, value of m corresponds to the numbers of building blocks (repeating units) in each polymer on average. In one embodiment, m is calculated by dividing Mn by the molecular weight of each building block. For example, the polymer P1-6 with Mn of 17449 Daltons has 23.3 building blocks in each polymer on average, and m is 23.3. In one embodiment, m has a value in a range of 20-300. In one embodiment, Mn of the polymer is in a range of 2,000-100,000 Daltons. In one embodiment, Mw of the polymer is in a range of 5,000-300,000 Daltons.

### Example 6—Preparation of Chemiresistive Sensor

The preparation of chemiresistive sensor comprised the following steps:
(a) Preparing the ink solution by dissolving the polymer synthesized in Example 5 (e.g., Polymer P1-6) in an organic solvent such as chloroform, chlorobenzene, dichlorobenzene, toluene, anisole or ketone in a concentration of 5-10 mg/mL, wherein the solution was warmed at 60° C. or until the polymer is fully dissolved;
(b) Printing the ink solution onto a sensor substrate by spin-coating, blade coating or inkjet printing techniques;
(c) Drying the ink solution at 100° C. to give a uniform thin film in 10-100 nm thick;
(d) Thermally annealing the thin film at 80° C.-250° C. for 30 minutes to remove the cleavable side chains from the polymer backbone;
(e) Once cooled to room temperature, submerging the sensor device in a dopant solution (such as iodine, pTSA, HCl, and HBr) and warming at 50° C.-80° C. for an additional 30 minutes. In one embodiment, the dopant can be pTSA or HCl. The content of the dopant within the complex ranges from 10-50 wt %.

The final sensor had a geometry as indicated in FIG. 3. The electrode channel length of the sensor ranged from 10 to 100 microns.

### Example 7—Analysis of Working Voltage of the Present Sensors

The sensor fabricated in Example 6 was operated at room temperature as a chemiresistive VOCs sensor with an operational voltage as low as 1 volt.

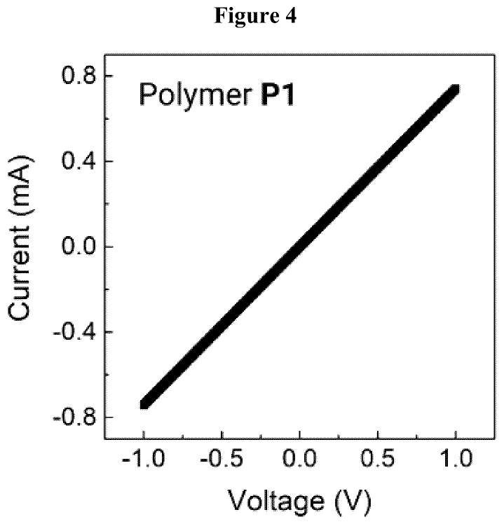

FIG. 4 shows the ohmic behavior of the polymer P1-6 before cleavage and doping as an example where the V/I curve is a straight line, indicating the suitable property serving as a chemiresistive material. As the resistance of the polymer is a constant, the resistance of the matrix-analyte complex changes when an analyte falls on the sensing matrix, and the signal response can be reflected by the change in current; therefore, the polymer can act as a chemiresistive material.

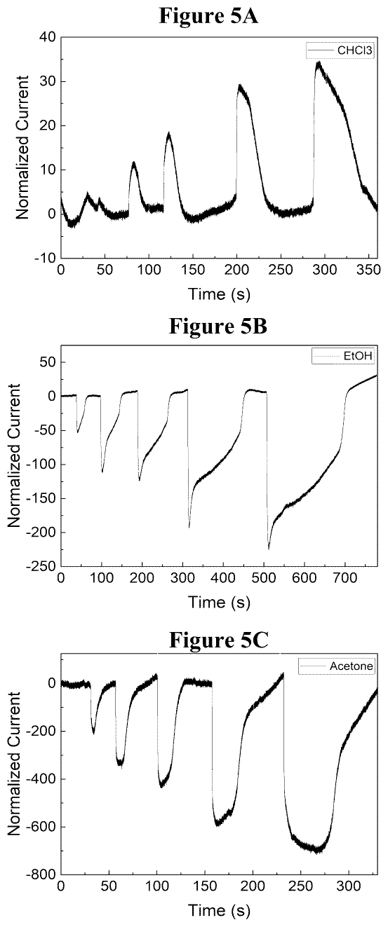

FIGS. 5A-5C show the current signals using the polymer, i.e., Polymer P1-6 as an example after side chain cleavage with pTSA as dopant (˜10% wt dopant in the polymer complex) as sensing matrix. A constant 1V operation voltage was applied and the different concentration of acetone vapor can be clearly shown on the graph. Proving the feasibility to operate the sensor made with the conducting polymer at low working voltage and therefore low power consumption.

### Example 8—Sensor Responses of P1-Sensing Material to Three Types of VOCs

FIGS. 5A-5C show example of current vs. time sensing curve of using P1 (i.e., polymer P1-6 as an example) after side chain cleavage with pTSA as dopant) (˜10% wt dopant in the polymer complex) as the sensing matrix using 1 volt operation voltage using a source/measure unit (SMU).

Chloroform, ethanol or acetone vapor of concentration 1, 5, 10, 25 or 50 ppm were introduced to the sensing matrix at different time intervals with a constant nitrogen carrier gas flow rate of 500 sccm (Standard Cubic Centimeters per Minute).

### Example 9—Principal Component Analysis

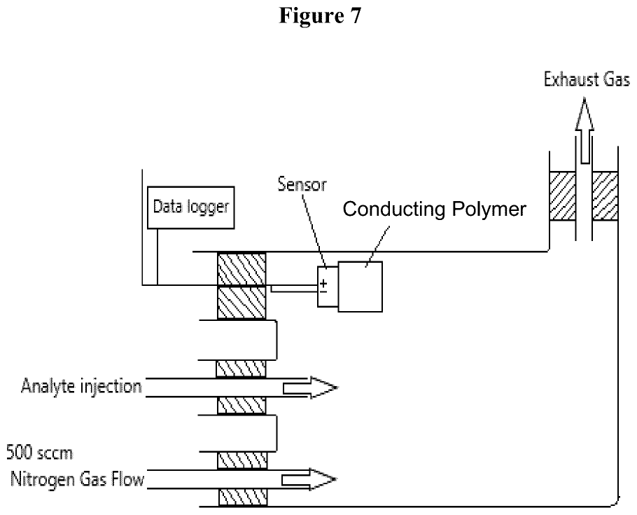

The principal component analysis was completed by an experimental setup as shown in FIG. 7 with the use of sensor assembled in Example 6 (i.e., Polymer P1-6 as an example) after side chain cleavage with pTSA as dopant) (˜10% wt dopant in the polymer complex) he analysis comprised following steps:
(a) Using a constant nitrogen flow rate of 500 sccm as carrier gas in a testing chamber;
(b) Using a current versus time scanning, introducing different VOCs to the sensing chamber, to obtain a current vs. time signal characteristic peak;
(c) For qualitative analysis of VOC, injecting a fixed molar concentration of gas into the test chamber, thereby resulting in one or more graphs, wherein the VOC may interact with the active sensing material molecularly in a different way. Such interaction includes interactions between VOC and the designed functionalities on the polymer chain such as hydrogen-bonds and weak intermolecular forces;
(d) Extracting signal features from the graphs, wherein said features are selected from the group including, signal intensity, elapsed time, peak area, peak height, peak FWHM, recovery time, and peak symmetry;
(e) Plotting the signal features in a graph with two or more dimensions, wherein each dimension (also termed as principal component) corresponds to one feature, wherein one compound or molecule has a unique coordinate in said graph; and
(f) Determining whether a target molecule exists in the sample.

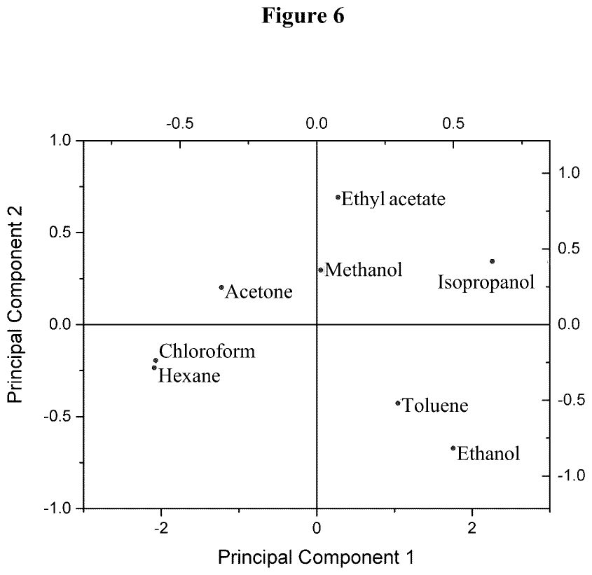

Principal component analysis plot of 8 different VOCs using the signal characteristic of the corresponding analytes is shown in FIG. 6. In one embodiment, the x-axis and the y-axis correspond to the eigenvalues generated from eigenvector-eigenvalue calculation of signals detected by sensors. In one embodiment, the eigenvector-eigenvalue calculation is conducted by a software. In one embodiment, the detected signals include but are not limited to peak height, peak width, peak area, signal intensity, elapsed time, slope. The plot indicated that different VOCs can be identified efficiently by analyzing their features (e.g. elapsed time, signal intensity, peak height, peak area slope and so on) and choosing two features with largest variance to separate different variables. Used in this example, Principal Component 1 (PC1—signal intensity) and Principal Component 2 (PC2—elapsed time representing the time taken from responding to recovering to normal) are the two PCs chosen to be applied in the plot in order to categorize different VOCs into different groups. The PCA is plotted based on the differences among each metabolite, i.e., metabolites with similar properties will cluster together in the PCA plot. The metabolites are categorized according to eigenvalue calculated from the signals detected.

A person of ordinary skill in the art can make the selection of signals for different analysis purpose. A person of ordinary skill in the art can further analyze or fine tune the PCA plot by means of artificial intelligence or pattern recognition software for more accurate results.

### Example 10—Sensor Responses of P1-Sensing Material to Three Types of VOCs

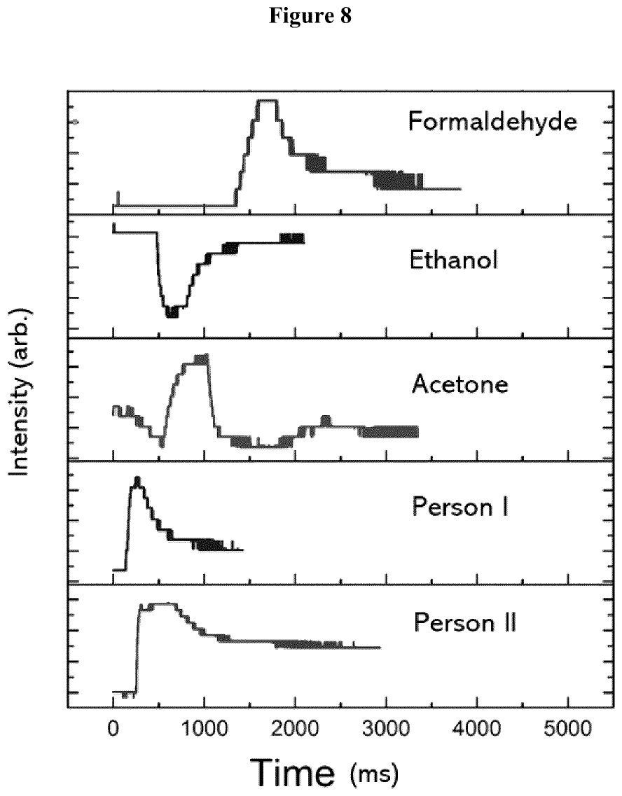

FIG. 8 represents specific signals produced using P1 (in this example, Polymer P1-6 as an example after side chain cleavage with pTSA as dopant) (˜10% wt dopant in the polymer complex) as the sensor material on acetone, ethanol, formaldehyde and breaths of two different persons. This experiment was conducted on a portable sensor using P1 as the sensor material, the results indicated that such prototype is capable of detecting gases under room temperature and requires low operating power. The small size of the prototype together with its long stability would facilitate the development of portable sensors for daily uses.

The experiments were conducted at room temperature (around 25° C.). The experiments comprised following steps:
(a) Using a voltage versus time scanning, introducing samples to the sensing chamber, i.e., formaldehyde, ethanol, acetone or breaths of Person I and Person II (collected by direct exhalation to the sensing chamber at room temperature without airflow control);
(b) Obtaining an arbitrary voltage vs. time signal characteristic peak, which can be further analyzed to determine the composition of tested gas or the breath of targeted person both qualitatively or quantitatively.

### Example 11—Stability Test Under Room Conditions

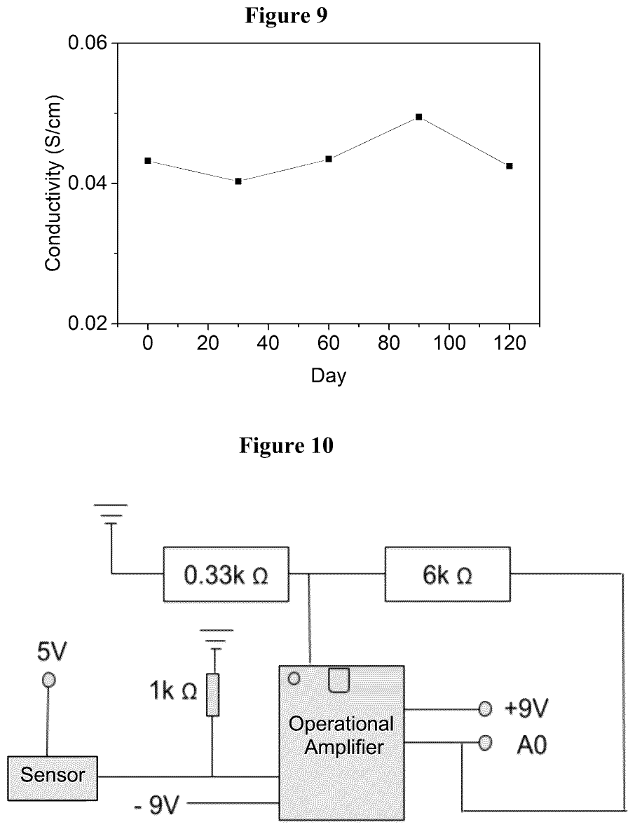

FIG. 9 shows the stability of the polymer P1-6 after side chain cleavage with pTSA as dopant) (˜10% wt dopant in the polymer complex) in sensor prepared in Example 6 stored in room condition. The conductivity of the polymer over at least 120 days of exposure to ambient air at room temperature was tested wherein the conductivity of the doped polymer in the same device was in a range of 0.04-0.05 S/cm and did not show a significant change demonstrating a good stability of the material in ambient condition.

### Example 12—Printable Conducting Polymer

The conductive ink is prepared by dissolving the polymer synthesized in Example 5 in an organic solvent such as chloroform, chlorobenzene, dichlorobenzene, toluene, anisole or ketone in a concentration of 1-10 mg/mL, wherein the solution is warmed at 60° C. or until the polymer is fully dissolved.

Then, the conductive ink can be casted onto an electrode substrate by means of various coating techniques such as spin coating, blade coating, drop casting and inkjet printing at low temperature processing environment under ambient condition to form a conductive crystalline thin film with edge-on orientation for sensor applications. By connecting to a printing system, the conducting polymer can be printed on a surface or circuit board smaller than 1 micrometer square, or other size depending on the cost and needs of the device.

### REFERENCES

1. Xinyu Zhang, Jeong-Soo Lee, Gil S. Lee, Dong-Kyu Cha, Moon J. Kim, Duck J. Yang, and Sanjeev K. Manohar, Macromolecules, 2006, 39 (2), 470-472.
2. Bruna M. Hryniewicz and Marcio Vidotti, ACS Applied Nano Materials 2018 1 (8), 3913-3924.
3. Muhammad Mumtaz, Emmanuel Ibarboure, Christine Labrugere, Eric Cloutet and Henri Cramail, Macromolecules 2008 41 (23), 8964-8970.
4. Jianhua Liu and Thuc-Quyen Nguyen, Material Matters 7.1.
5. Weiwei Li, Koen H. Hendriks, Martijn M. Wienk, and René A. J. Janssen, Diketopyrrolopyrrole Polymers for Organic Solar Cells, Acc. Chem. Res., 2016, 49 (1), 78-85.
6. Eric Daniel Glowacki, Halime Coskun, Martin A. Blood-Forsythe, Uwe Monkowius, Lucia Leonat, Marek Grzybowski, Daniel Gryko, Matthew Schuette White, Alan Aspuru-Guzik, Niyazi Serdar Sariciftci, Hydrogen-bonded diketopyrrolopyrrole (DPP) pigments as organic semiconductors, Organic Electronics, December 2014, (12), 3521-3528.
7. A. G. Macdiarmid, J. C. Chiang, A. F. Richter A., and J. Epstein, Synthetic Metals, 18 (1987) 285-290.
8. Schubert, E. F. (2005). Doping in III-V Semiconductors. pp. 241-243. ISBN 978-0-521-01784-8.
9. Hua Bai and Gaoquan Shi, Gas Sensors Based on Conducting Polymers, Sensors 2007, 7(3), 267-307.
10. Marek Grzybowski, Daniel T. Gryk, Diketopyrrolopyrroles: Synthesis, Reactivity, and Optical Properties, Advanced Optical Materials, 2015, 3(3), 280-320.
11. Artur Rydosz, Sensors for Enhanced Detection of Acetone as a Potential Tool for Noninvasive Diabetes Monitoring. Sensors 2018, 18(7), 2298.
12. Marco Righettoni, Antonio Tricoli and Sotiris E. Pratsinis, Si:WO(3) Sensors for highly selective detection of acetone for easy diagnosis of diabetes by breath analysis. Anal Chem. 2010 May 1; 82(9), 3581-7.
13. Minh Tdo C, Blake D R, Galassetti P R, The clinical potential of exhaled breath analysis for diabetes mellitus. Diabetes Res Clin Pract. 2012 August; 97(2), 195-205.
14. Yixia Zhang et al., Identification of Volatile Biomarkers of Gastric Cancer Cells and Ultrasensitive Electrochemical Detection based on Sensing Interface of Au—Ag Alloy coated MWCNTs, Theranostics. 2014; 4(2): 154-162.

What is claimed is:

1. A polymer represented by a formula selected from the group consisting of 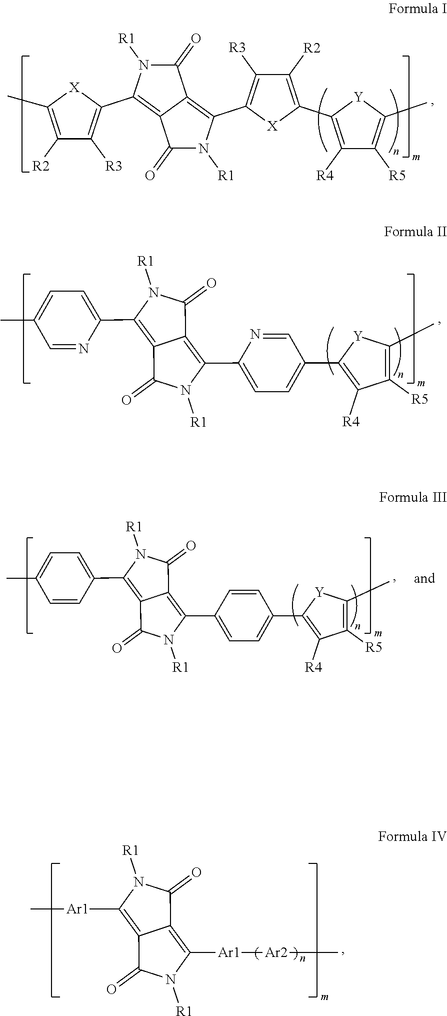
wherein R1 is selected from the group consisting of hydrogen, acyl, carboxyl, alkyl, alkenyl, alkynyl, hydroxyalkyl, halogen, haloalkyl, ester, ether, aldehyde, ketone, carboxylic acid, azo, Z-alkyl, and metal containing organometallic complex, wherein Z is selected from the group consisting of cyclic ether, amine, amide, imine, azide and sulfonyl,
wherein each of the R2, R3, R4 and R5 is independently selected from the group consisting of hydrogen, acyl, carboxyl, alkyl, alkenyl, alkynyl, alkoxy, hydroxyalkyl, halogen, haloalkyl, ester, ether, cyclic ether, amine, amide, imine, aldehyde, ketone, carboxylic acid, azide, azo, amide functionalized hydrocarbon chain, and metal containing organometallic complex,
wherein X and Y are independently selected from oxygen, sulphur and selenium;
wherein each of Ar1 and Ar2 is independently selected from the group consisting of naphthalenyl, anthracenyl, phenanthracenyl, triphenylene, pyrenyl, thienothiophenyl, dithienothiophenyl and benzodithiophenyl, and
wherein n ranges from 1-5, and m is at least 3.

2. The polymer of claim 1, wherein R1 is selected from the group consisting of hydrogen, acyl, long chain alkyl, long chain alkenyl and long chain alkynyl, R2 and R3 are independently selected from the group consisting of hydrogen, straight alkyl chain and branched alkyl chain, and R4 and R5 are independently selected from the group consisting of hydrogen, straight alkyl chain, branched alkyl chain, ester, ether, and amide functionalized alkyl, alkenyl, and alkynyl chain.

3. The polymer of claim 1, wherein n ranges from 1-5, and m is in a range of 10-300.

4. The polymer of claim 1, wherein Mn of said polymer is in a range of 2,000-100,000 Daltons.

5. A method of synthesizing the polymer of claim 1, comprising the steps of
(a) Preparing an acceptor building block comprising a diketopyrrolopyrrole (DPP),
(b) Functionalizing a pyrrole nitrogen of the acceptor building block;
(c) Treating the product of step (b) with a brominating reagent, thereby obtaining a brominated product;
(d) Preparing a donor building block capable of undergoing Stille-type reaction with a brominated compound; and
(e) Allowing the brominated product and the donor building block to undergo Stille-type reaction, thereby obtaining the polymer.

6. A method of preparing a thin film on a substrate for a sensing component, comprising the steps of:
a) Printing a solution of the polymer of claim 1 in an organic solvent onto a substrate;
b) Drying the printed solution, leading to a dried film on the substrate;
c) Thermally annealing the dried film; and
d) Submerging the dried film on the substrate in a solution of dopant, thereby obtaining the thin film deposited onto the substrate.

7. The method of claim 6, wherein the organic solvent is selected from the group consisting of chloroform, chlorobenzene, dichlorobenzene, toluene, anisole or ketone and the solution of the polymer has a concentration of 1-10 mg/mL.

8. The method of claim 6, wherein the printed solution is dried at 100° C.

9. The method of claim 6, wherein the dried film is thermally annealed at 80° C.-250° C. for 10-60 minutes to remove the cleavable side chains R1 from the polymer and form a crosslinked structure which makes the thin film insoluble, stable, durable and chemical resistant.

10. The method of claim 9, wherein R1 is acyl, carboxyl, methyl or ethyl.

11. The method of claim 6, wherein the dopant is selected from the group consisting of hydrofluoric acid, hydrochloric acid, hydrobromic acid, hydroiodic acid, p-toluenesulfonic acid (pTSA), acetic acid, nitric acid, sulfuric acid, phosphoric acid, tetracyanoquinodimethane (TCNQ) 7,7,8,8-tetracyano-2,3,5,6-tetrafluoroquinodimethane (F4TCNQ) and elemental iodine.

12. The method of claim 6, wherein the dried film on the substrate is submerged in the solution of dopant at room temperature followed by an elevated temperature at 50° C.-80° C.

13. The method of claim 6, wherein the thin film has a thickness of 10-100 nm.

14. A device for detecting molecules, comprising a sensing component comprising two or more electrodes on a substrate deposited with a thin film prepared by claim 6.

15. The device of claim 14, wherein the device further comprises one or more of the following:
(a) Dust filters;
(b) Moisture absorption filters;
(c) Sample concentrator;
(d) Gas flow rate meter; and
(e) Liquid flow meter.

16. The device of claim 14, wherein the device can be operated under room temperature, pressure and humidity and requires a low working voltage.

17. The device of claim 14, wherein the working voltage is 10 mV-10V.

18. The device of claim 14, wherein the polymer of the sensing component has a shelf life of at least 4 months under room temperature, pressure, and humidity.

19. The device of claim 14, wherein said device can detect an analyte with a limit of detection of about 1000 ppb.

20. A method of detecting target molecules in a gas or liquid sample using the device of claim 13, comprising the following steps:
(a) Introducing the sample to the device and conducting a current or voltage scanning, obtaining one or more graphs, wherein the graphs can be a plot of current vs time or voltage vs time;
(b) Extracting signal features from said graphs, wherein said features are selected from the group consisting of signal intensity, elapsed time, peak height, peak area, peak FWHM, recovery time, and peak symmetry;
(c) Projecting said features to a map with two or more dimensions, wherein each dimension corresponds to one feature, and the position of a specific compound in said map is unique; and
(d) Determining whether the target molecules are present in the sample.

21. The method of claim 20, wherein the map is a two-dimensional map, one dimension represents elapsed time, and the other dimension represents signal intensity.

22. The method of claim 20, wherein the target molecules are volatile organic compounds (VOCs) and said VOCs are selected from the group consisting of methanol, ethanol, propan-1-ol, isopropanol, acetone, acetic acid, chloroform, hexane, ethyl acetate, toluene, chlorobenzene, diethyl ether, tetrahydrofuran, 1,2-dioxane, 1,4-dioxane, ethyl formate, butanone, acetonitrile, benzene, and carbon disulphide.

23. The method of claim 20, wherein the molecules are selected from the group consisting of formaldehyde, carbon monoxide, ammonia, hydrochloric acid, chlorine, sulphur oxides, and sulfuric acid.

24. The method of claim 20, wherein the method further determines the quantity or concentration of the target molecules in the sample.
